(19) United States
(12) Patent Application Publication
(10) Pub. No.: US 2026/0345967 A1
(43) Pub. Date: Nov. 5, 2026

(54) ENHANCED DUAL LUMEN MICROCATHETER BALLOON SYSTEM

(71) Applicant: Emerald Neurovascular, Hunts Point, WA (US)

(72) Inventor: Stephen Monteith, Hunts Point, WA (US)

(21) Appl. No.: 19/660,330

(22) Filed: Apr. 28, 2026

Related U.S. Application Data
(60) Provisional application No. 63/797,333, filed on Apr. 30, 2025.

(51) Int. Cl. A61M 25/10; A61M 25/00

(52) U.S. Cl. CPC A61M 25/10; A61M 25/0021; A61M 2025/0042; A61M 2025/1075; A61M 2205/0222; A61M 2205/0238; A61M 2205/32; A61M 2205/50; A61M 2210/0693; A61M 2210/12

(57) ABSTRACT

A microwire based endovascular micro-balloon system as configured is easily navigated into the distal vessel (such as the distal vertebral artery, internal carotid artery, or external carotid artery) without causing the access catheter (for example a SIMMONS type catheter) to herniate out of the target vessel and into the aorta. As taught herein the micro-balloon is inflated with contrast to allow visibility under fluoroscopy. Following this the larger bore guide or intervention catheter is advanced along the microwire / micro-balloon system which is secured by the balloon being inflated once the guide catheter is advanced distally the balloon can be deflated and removed.

### CROSS REFERENCE TO RELATED APPLICATIONS

This application claims priority from U.S. Provisional Application No. 63/797,333 filed 04/30/2025 by the instant assignee, EMERALD NEUROVASCULAR (Hunts Point, WA 98004).

### FIELD OF THE DISCLOSURES

The inventive disclosures relate to providing a balloon system in a dual lumen microcatheter for anchoring inflation of the balloon with liquid contrast through a separate inflation/deflation port, to permit a microwire for navigation through the secondary lumen in the microcatheter.

As described above, an enhanced balloon anchor system allows ingress and egress to newly secured over-the-wire targets, as detailed and claimed below.

### BACKGROUND OF THE DISCLOSURES

The instant inventions relate to the field of endovascular interventions to evaluate, address, treat, update and otherwise provide clinically relevant treatment options. In particular, using both known and newly improved access techniques comprising trans-radial, trans- carotid, or other approaches optimize applications of the instant systems, while artisans readily understand that these systems likewise benefit trans-femoral access, traditionally used and with which the instant inventions are configured to work.

### OBJECTS & SUMMARY OF THE INVENTIONS

Briefly stated, balloons used in endovascular procedures function as anchors to improve clinical applications, particularly above the aortic arch and likewise with trans-radial, trans-femoral and other approaches.

According to embodiments, the present disclosure provides a dual lumen microcatheter balloon system, and features the improvement, comprising, in combination, an enhanced system featuring an ultra-compliant balloon; the balloon being coated in frictionless or higher friction coating to aid anchoring inflation of the balloon with liquid controls; a separate inflation/deflation port from the guidewire port; and, said guidewire port permits 0.014 inch diameter microwire for navigation through secondary lumen in microcatheter. The anchoring balloon may be completely occlusive in nature or may be non occlusive and allow blood to flow past it while it is fully inflated. This is achieved by way of having a cross section that allows blood flow past the balloon while the balloon is inflated. Examples of this may include a bow-tie shaped balloon in cross section or a kidney bean shaped balloon in cross section.

The anchoring balloon is often used to allow the advancement of larger bore catheters over the balloon anchor system. The anchoring balloon may be used to act as a buddy system to hold the access system in place as other devices are advanced toward the treatment target through the same access catheter. Devices used for the treatment of intracranial or extracranial pathologies may be stiff such as stents or other devices used in the treatments of intracranial pathology. The balloon anchor system allows the delivery system to be anchored in position as the devices are advanced toward the treatment target.

According to embodiments, there is an application providing real time visual access to said therapy. Artisans understand that this includes both hardware and software along with animated graphical user interfaces to support Clinician access to the resultory data from use of the enhanced system.

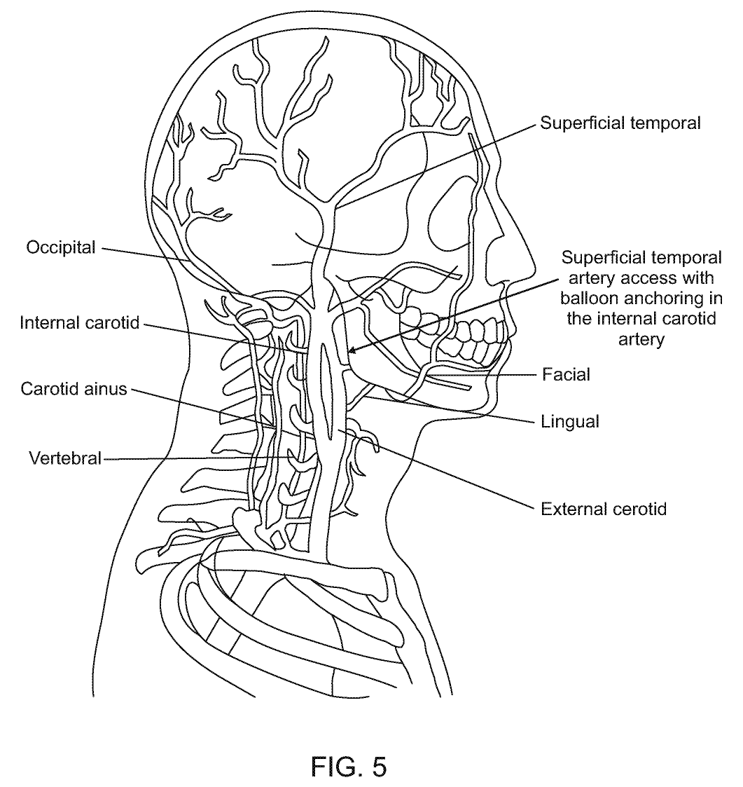

According to embodiments, there is a product by the process as shown in FIG. 5, being emplaced system showing internal carotid anchoring along with MMA access as shown and claimed throughout.

### BRIEFDESCRIPTIONOFTHEFIGURES

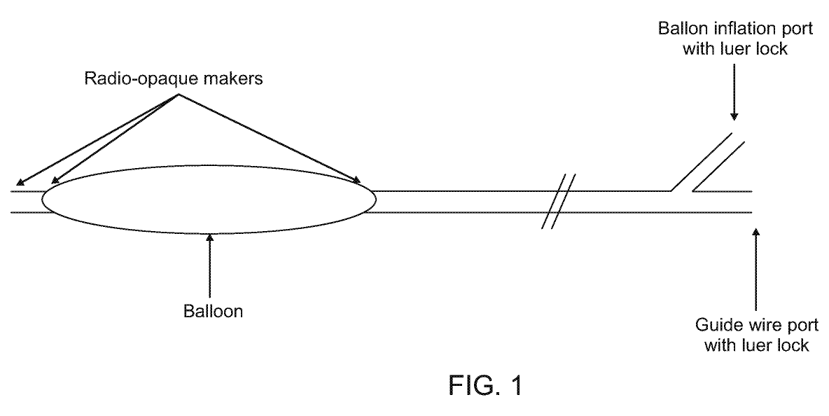

FIG. 1 shows an exemplary but not limiting system with relative positioning of aspects of system elements;

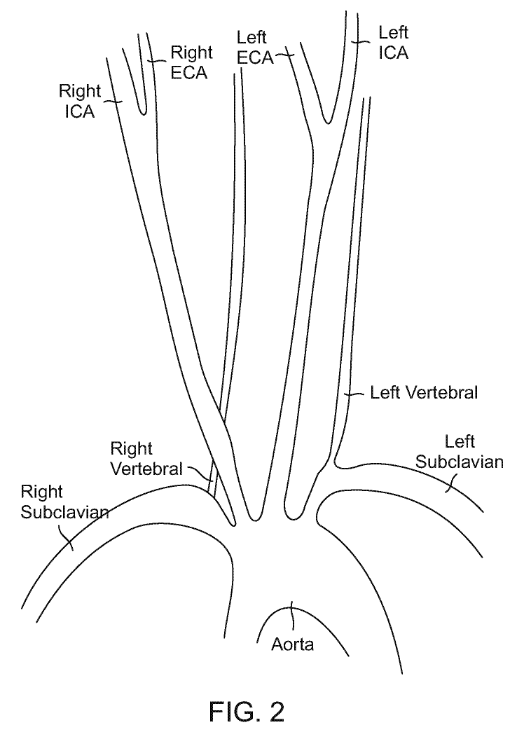

FIG. 2 details gross morphology of human anatomy proximate to the aortic arch;

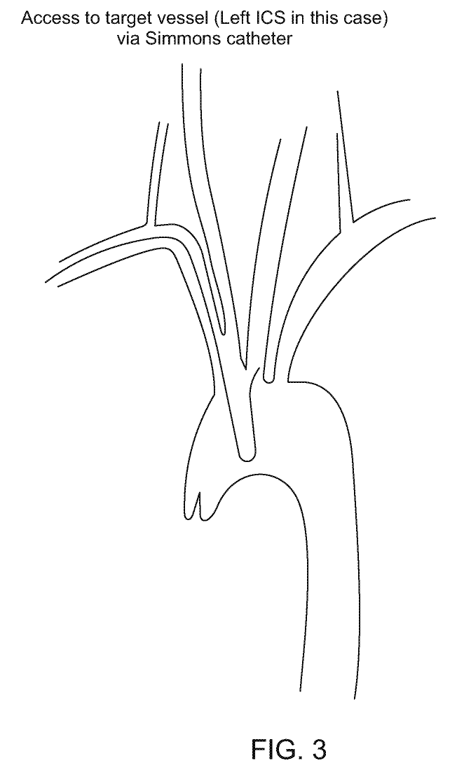

FIG. 3 shows an example of left internal carotid artery access with the teachings of the present invention;

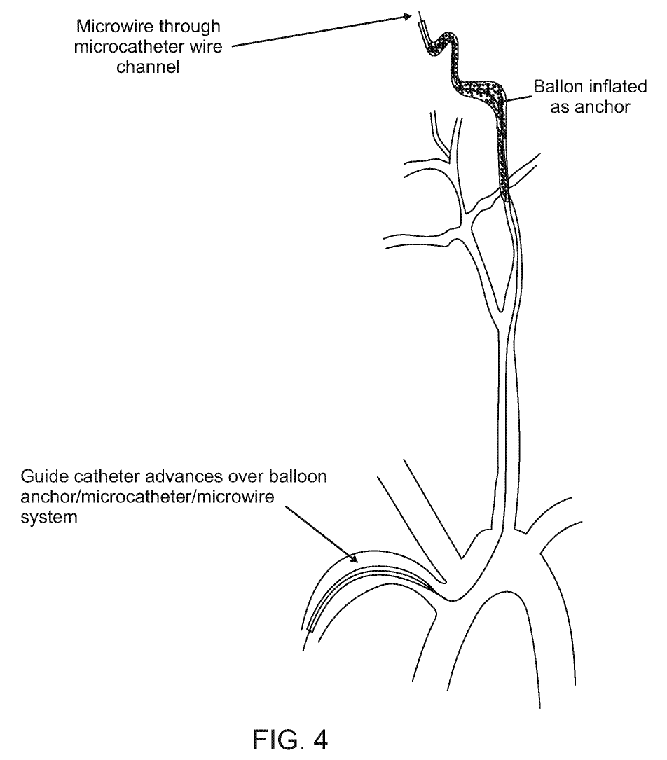

FIG. 4 illustrates a balloon inflated in the left distal ICA as an example of one application of the systems and methods of the present disclosure and inventions; and,

FIG. 5 shows products by the process of using the present invention, namely an emplaced system showing internal carotid anchoring along with MMA access as shown and claimed throughout.

### DETAILED DESCRIPTIONS OF THE INVENTIONS

The present inventor has discovered a system which supports the delivery of endovascular therapies including emplaced system showing internal carotid anchoring along with MMA access as shown and claimed throughout. One preferred mechanism is comprised of a process used to advance large bore catheters into a distal vasculature leading to the head and brain, without falling into the aortic arch. The device could be used in other locations in the body were anchoring the delivery system would be required.

The following patents are incorporated by reference as if fully set forth herein in their entirety herein, and comprise the closest prior art developed prior to the advent of the instant teachings: U.S. Letters Patent Nos. 11,224,449; 11,026,692; 11,154,690; 10,736,729; 9,339,368; 8,632,877; 7,648,497; and 6,093,194, along with their respective reference publications.

Referring now to all of the figures which are presented in schematized form for clarity of elements shown and particularly with respect to FIG. 1, this drawing shows a cartooned schematic view of typical systems with respect to balloon inflation ports and guidewire ports having lever locks, along with a subject balloon and radiopaque markers. By way of illustrative but not-limited exemplary embodiments – those skilled readily understand that the instant systems contemplate balloon lengths of at least 5, 10, or 15 cm; diameters between about 4 mm and 10 mm with an ultra-compliant balloon.

Referring now to FIG. 2, it is respectfully submitted that the complexity of the anatomy above the aortic arch creates daunting navigational issues for catheters. The instant balloon catheters anchor by being coated with frictionless or higher friction coatings; being dual lumen systems emplaced allowing inflation with contrast visualized under fluoroscopy. Separating inflation/deflation ports from guidewire ports permits 0.014 inch diameter microwire for navigation through secondary lumen in microcatheter.

Referring now to all the Figures and the Appendix expressly incorporated by reference and in particular to those latter figures, specific points of invention include the exemplary FIGS. 3 and 4 showing the novelty and efficacy of the present invention, albeit in cartooned schematics.

In particular FIG. 3 shows access to the left ICA, with a Simmons® brand of catheter, while FIG. 4 shows aspects of the procedure according to the instant teachings, namely, as claimed below, the microwire is traveled through the microcatheter wire channel, the balloon is inflated as an anchor after the guide catheter advanced over balloon anchor/microcatheter microwire system.

FIG. 4 likewise further shows how system works with hydrophilic coating on balloon and catheter, markers for tip of catheters as well as proximate and distal balloon ends, 150 cm microcatheter (up to at least about 300 cm plus) and 0.014 inch diameter with body over the section across aortic arch.

Referring still to all of the figures and in particular to FIG. 5, the present inventions as claimed and shown are embodied wherein an enhanced system having the minimum required elements depicted in the claims is represented to those skilled in the art. Namely, wherein unexpectedly the larger bore or internal catheter is advanced along the herein disclosed system which is secured by said enhanced dual lumen catheter introduced balloon, functioning as an anchor, and secured by said balloon being inflated, within the vessel illustrated or any others disclosed.

As used herein, including the appended claims, the singular forms of words such as "a," "an," and "the" include their corresponding plural references unless the context clearly dictates otherwise.

All references cited herein are incorporated by reference to the same extent as if each individual patent, and published patent application, as well as figures, drawings, sequence listings, compact discs, and the like, was specifically and individually indicated to be incorporated by reference. The present invention is not to be limited by compositions, reagents, methods, diagnostics, laboratory data, and the like, of the present disclosure. Also, the present invention is not to be limited by any preferred embodiments that are disclosed herein.

Various objects, features, aspects, and advantages of the inventive subject matter will become more apparent from the following detailed description of preferred embodiments, along with the accompanying drawing figure.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles "a" and "an," as used herein in the specification and in the claims, unless clearly indicated to the contrary, should be understood to mean "at least one." The phrase "and/or," as used herein in the specification and in the claims, should be understood to mean "either or both" of the elements so conjoined, i.e., elements that are conjunctively present in some cases and disjunctively present in other cases. Other elements may optionally be present other than the elements specifically identified by the "and/or" clause, whether related or unrelated to those elements specifically identified, unless clearly indicated to the contrary.

Reference throughout this specification to "one embodiment" or "an embodiment," means that a particular feature, structure, or characteristic described in connection with the embodiment is included in at least one embodiment. Thus, appearances of the phrases "in one embodiment", or "in an embodiment" in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to "one embodiment," "an embodiment," or similar language means that a particular feature, structure, or characteristic described in connection with the embodiment is included in at least one embodiment of the present invention. Thus, appearances of the phrases "in one embodiment," "in an embodiment," and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term "about." Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms "a," "an," "the" and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., "such as") provided herein is intended merely to better illuminate the invention, and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term "consisting of' excludes any element, step, or ingredient not specified in the claims. The transition term "consisting essentially of' limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An 1/0 mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term "machine-readable medium" should be taken to include a single medium or multiple media (e.g., a centralized or distributed database, and/or associated caches and servers) that store the one or more sets of instructions. The term "machine-readable medium" shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, "cloud" storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. In a dual lumen microcatheter balloon system, the improvement, comprised of, in
combination:
at least an enhanced system featuring an ultra-compliant balloon;
the balloon being coated in frictionless or higher friction coating to aid anchoring inflation of the balloon with liquid controls;
at least two ports, namely, a separate inflation/deflation port from a guidewire port;
whereby;
said guidewire port permits 0.014 inch diameter microwire for navigation through secondary lumen in microcatheter;
a stiffened guidewire body effective to be positioned to access the aortic arch; and
wherein advancement of larger bore catheters into the distal vasculature is facilitated via balloon anchoring in proximate vessels such as the internal Carotid artery which is secured by the balloon being inflated therein.

2. The system of claim 1, further comprising: at least a hydrophilic coating on balloon and catheter.

3. The system of claim 2, being markers for tip of catheter and distal and proximate ends of the balloon.

4. The system of claim 3, further comprised of at least a working microcatheter length of between at least about 138 and approximately 161 cm.

5. The system of claim 4, further comprising a 300-330 cm version.

6. The system of claims claim 5, wherein the stiffened guidewire body enables wherein large bore catheters to navigate more efficiently into the brain secured by a properly emplaced balloon being inflated.

7. The system of claim 6, said stiffened guidewire providing access to vessels further comprising the subclavian artery, the right and left vertebral artery, the right internal carotid artery (ICA), and right external carotid artery (ECA), the left external carotid artery (ECA), and the left internal carotid artery (ICA).

8. A process using the system of claim 7, further comprising the steps of: using a trans-radial artery access approach.

9. The processes of claim 8, wherein said dual lumen microcatheter comprises an inner guidewire lumen for traveling over a wire, and an outer balloon lumen.

10. The processes of claim 9, wherein at least two separate lever locked ports provide balloon inflation and wire portage respectively at a proximal end.

11. The processes of claim 10, whereby the access path comprises at least about half a dozen vessels further comprising the subclavian artery, the right and left vertebral artery, the right internal carotid artery (ICA), and right external carotid artery (ECA), the left external carotid artery (ECA), and the left internal carotid artery (ICA).

12. The process of claim 11, wherein delivery desiderata include managing catheters being advanced from the subclavian into any said artery without allowing falling or dropping into the Aortic arch which negates their efficacy.

13. The process of claim 12, preventing herniation out of target vessels into the aorta.

14. The processes of claim 13, wherein said microwire/micro-balloon system is inflated with contrast, under fluoroscopy.

15. The processes of claim 14, wherein unexpectedly the larger bore or internal catheter is advanced along the herein disclosed system which is secured by said enhanced dual lumen catheter introduced balloon, functioning as an anchor, and secured by said balloon being inflated.

16. The process of claim 15, wherein access other than radial is used.

17. A product by the process of claim 16, said ballon catheter being emplaced within internal carotid artery enabling access to the MMA, as shown in FIG. 5.

18. An application (computer app) providing real-time visual access to said therapy; and storing resultory data and allowing it to be used for further data refinement within the system.

19. The computer app of claim 18, said refined data comprised of human gated or mediated AI.

20. The computer app of claim 19, comprised of an indication that the product which is secured by the balloon being inflated has been properly emplaced.
